(19) United States
(12) Patent Application Publication
(10) Pub. No.: US 2012/0015390 A1
(43) Pub. Date: Jan. 19, 2012

(54) Methods for the Detection and Treatment of Aberrant Prion Disease

(75) Inventors: Christiaan Roelant, Leuven (BE); Kenny De Meirleir, Mechelen (BE)

(73) Assignee: PROTEA BIOPHARMA N.V., Zellik (BE)

(21) Appl. No.: 13/259,404

(22) PCT Filed: Mar. 26, 2010
(86) PCT No.: PCT/EP2010/054006

§ 371 Date: Sep. 23, 2011

(30) Foreign Application Priority Data
Mar. 27, 2009 (EP) 091564674

(51) Int. Cl. C12Q 1/26; C12Q 1/02

(52) U.S. Cl. 435/25; 435/29

(57) ABSTRACT

The invention relates to methods and tools for detecting and treating patients suffering from aberrant prion functioning or Aberrant Prion Disease (APD), methods for determining the presence of aberrant prion functioning-inducing agents in a sample and to methods for determining the appropriate therapy for a patient having symptoms of aberrant prion functioning based on detecting aberrant NADH oxidase activity.

### FIELD OF THE INVENTION

The invention relates to methods and tools for detecting and treating aberrant prion functioning and detecting and treating patients suffering from Aberrant Prion Disease (APD), methods for determining the presence of APD-inducing agents in a sample and to methods for determining the appropriate therapy for a patient having symptoms of APD.

### BACKGROUND

Cell surface NADH oxidase or Ecto-Nox proteins are proteins, located at the cell surface, which are involved in time-keeping and cell-growth. They are described to have both hydroquinone (NADH) oxidase and protein disulfide-thiol interchange activities which alternate within a 24 minute period (Kim et al. 2002, J. Biol. Chem. 277:16441-16447). A constitutively activated form designated tNOX has been described as being associated with cancer. WO 9526743 generally discloses the use of NADH oxidase as a target in the diagnosis and therapy of cellular disease states, particularly neoplastic and virally infected cells, and in the screening for active agents for the treatment of such diseased states and overcoming multiple drug resistance. U.S. Pat. No. 5,569,673 describes the use of N-acylated catecholmethylamines, particularly the monomethyl ether, as inhibitors of NADH oxidase activity associated with neoplastic cells.

A large number of individuals suffer from a pathological condition which is generally characterized by chronic fatigue and a lack of energy. A number of these individuals are diagnosed as suffering from Chronic fatigue syndrome (CFS), also known as Myalgic Encephalomyelitis (ME), a disease with an unknown etiology generally characterized by persistent or relapsing fatigue interfering with normal function. The symptoms of chronic fatigue and lack of energy however are sometimes considered to be indicative of or attributable to mononucleosis, anaemia, an infectious disease, as a side-effect of chemotherapy or related to other diseases or stress factors.

The diagnosis of Chronic Fatigue Syndrome has been the subject of much debate and is still not recognized by a number of health insurance providers. Its unknown aetiology and the variety in the nature and severity of clinical symptoms suggest that it in fact covers different underlying physiological phenomena. A set of diagnostic criteria was developed by the U.S. Centres for Disease Control and Prevention in 1994, the most common being severe mental and physical exhaustion which is unrelieved by rest. While these diagnostic criteria require that the symptoms must not be caused by other medical conditions, this is in fact often difficult to determine with certainty. The fact that the symptomatic criteria do not identify patients suffering from the same physiological problems is underscored by the fact that most therapies or remedies that have been developed for CFS or its symptoms are effective in only 50-70% of the patients treated (Treatment of Chronic fatigue Immune Dysfunction Syndrome Using a Kutapressin complex. Derek Enlander M.D. (in press)

Accordingly, there is a need for additional criteria allowing the further characterization of patients suffering from chronic fatigue and lack of energy so as to improve existing therapy and the development of new, more effective therapeutics.

Mammalian liver extract has been used for the treatment of a wide range of diseases. It has been commercialized under the names of Kutapressin® and Nexavir®. U.S. Pat. No. 5,055,296 describes the use of a mammalian liver extract for the treatment of viral infections and chronic fatigue syndrome. The extract is described to be thermostable, acetone-insoluble and soluble in water. Chemical analysis of the liver extract revealed at least five polypeptides of which one was found to have bradykinin-potentiating activity. In U.S. Pat. No. 5,334,395, which relates to the use of a mammalian liver extract in the treatment of Epstein Barr Virus (EBV) infection, 9 peptides are identified in the Kutapressin extract as having angiotensin converting enzyme inhibitory activity. One of these peptides is demonstrated to be capable of inhibiting EBV infection in vitro. The exact nature of these peptides or their mechanism of action is not disclosed.

### SUMMARY OF THE INVENTION

The present invention is based on the observation that a number of diseases are characterized by aberrant ecto-nox functioning. Such diseases can generally be referred to as aberrant prion diseases.

Further, the present invention is based on the observation that aberrant ecto-nox or membrane NADH oxidase activity is at the basis of a number of symptoms including, but not limited to fatigue and that aberrant ecto-nox functioning is at the basis of diseases characterized by chronic fatigue, such as Chronic Fatigue Syndrome or CFS.

One aspect of the invention provides in vitro methods of determining whether or not a patient is suffering from aberrant ecto-nox functioning. More particularly, the methods relate to determining whether or not a patient is suffering from aberrant functioning of constitutive ecto-nox proteins. Indeed, the identification of a patient suffering from aberrant ecto-nox functioning not only allows the identification of an actual physiological disfunctioning (in cases where this would be questionable), but moreover makes it possible to consider whether this aberrant functioning can be treated. Accordingly, in particular embodiments the invention relates to methods for determining whether or not a patient is suffering from aberrant ecto-nox functioning comprising: (a) contacting a cell-containing sample of the patient with an NADH solution and a colorimetric or luminometric substrate, and detecting NADH oxidase activity in the sample.

In particular embodiments the invention relates to methods for diagnosing a patient with Aberrant Prion Disease, which methods comprise:
(a) contacting a cell-containing sample of the patient with an NADH solution and a colorimetric or luminometric substrate, and
(b) detecting NADH oxidase activity.
   In further particular embodiments the methods referred to herein further comprise the step of comparing NADH oxidase activity to that of a control sample and/or involve contacting the sample with a hypotonic NADH solution and/or an isotonic NADH solution.

According to particular embodiments, methods for determining whether or not a patient is suffering from aberrant ecto-nox functioning and/or methods for diagnosing a patient with Aberrant Prion Disease are provided which comprise:
(a) contacting a cell-containing sample of the patient with an NADH hypotonic and/or a NADH isotonic solution, and
(b) comparing the effect of the NADH hypotonic solution and/or the NADH isotonic solution on the cells to the effect of the NADH hypotonic solution and/or the NADH isotonic solution on a control sample.

Most particularly, these methods can be used for determining the susceptibility of a patient for the treatment with an agent capable of normalizing the aberrant ecto-nox functioning, more particularly with an ecto-nox modifying agent.

Yet a further aspect of the present invention provides methods for determining the presence of (aberrant) ecto-nox proteins in a sample, more particularly a biological sample of a patient, which methods comprise:
(a) contacting the sample with a colorimetric or luminometric substrate and NADH, and
(b) detecting NADH oxidase activity, wherein the intensity of the color or light is indicative of the presence of (aberrant) ecto-nox proteins in a sample.

In particular embodiments, these methods comprise prior to step (a) the step of subjecting the sample to denaturing conditions and determining the effect of said denaturing conditions, more particularly subjecting the sample to heating. In further particular embodiments, the sample is contacted with NADH under isotonic or hypotonic conditions and the effect of these conditions on the NADH oxidase activity is determined.

In particular embodiments these methods are used for determining the presence of aberrant ecto-nox proteins in a patient sample. In further particular embodiments, these methods are used for diagnosing Aberrant Prion Disease or a form of CFS characterized by the presence of aberrant prions.

In yet a further aspect the invention relates to methods of screening for therapeutic agents, which methods comprise, contacting a sample of a patient which has been identified to have aberrant ecto-nox functioning with a compound of interest and determining whether or not the compound is capable of reducing or inhibiting aberrant ecto-nox functioning in the sample. Such methods allow for the identification of one or more compounds which can be used in the treatment of the patient from which the sample originates.

A further aspect of the invention relates to compositions comprising an ecto-nox protein modifying agent for use in the treatment of patients characterized by aberrant ecto-nox functioning, more particularly for use in the treatment of an aberrant prion disease. In particular embodiments, the compositions according to the invention are suitable for use in the treatment of a patient suffering from chronic fatigue and characterized as having aberrant prion functioning.

In particular embodiments, compositions comprising one or more ecto-nox protein modifying agents are used, wherein the ecto-nox protein modifying agent is selected from the group consisting of:
a molecule having NADH oxidase activity capable of competing with the ecto-nox protein,
a molecule capable of inhibiting the association of an ecto-nox protein with the plasma membrane, and
an NADH oxidase inhibitor.

In further particular embodiments, the composition comprises constitutive membrane NADH oxidase. In particular embodiments the composition comprises a processed tissue extract. Alternatively, the composition is not a processed tissue extract but a purified fraction comprising constitutive membrane NADH oxidase.

The present inventors have identified that the effect of processed tissue extract described in the prior art on Chronic Fatigue Syndrome (CFS) is attributable to the presence of membrane NADH oxidase in these extracts. Accordingly it is submitted that identifying patients diagnosed with CFS as characterized by aberrant prion functioning or having Aberrant Prion Disease will allow a more efficient treatment of this patient group. Thus, according to particular embodiments, compositions comprising constitutive membrane NADH oxidase are provided for the treatment of CFS patients characterized by aberrant prion functioning or APD. In addition, the invention provides compositions comprising membrane NADH oxidase, other than processed tissue extracts, for use in the treatment of patients suffering from CFS, more particularly patients suffering from chronic fatigue or CFS characterized by aberrant prion functioning or APD.

In particular embodiments the compositions for use in the treatment of patients having aberrant prion functioning, such as patients having CFS comprise one or more ecto-nox protein modifying agents selected from the group consisting of:
a molecule having NADH oxidase activity capable of competing with the ecto-nox protein,
a molecule capable of inhibiting the association of an ecto-nox protein with the plasma membrane, and
an NADH oxidase inhibitor.

In further particular embodiments, the composition comprises one or more prion-like molecules as an active agent. Most particularly, the prion-like molecule is a membrane NADH oxidase.

In particular embodiments the ecto-nox protein modifying agent is a molecule capable of inhibiting the association of an ecto-nox protein with the plasma membrane, more particularly a solvent. Particular embodiments include DMSO.

Yet a further aspect of the invention relates to a membrane NADH oxidase for use in the treatment of a patient characterized by aberrant prion functioning. More particularly the patient has Chronic Fatigue syndrome or another disease which is an Aberrant Prion Disease. In particular embodiments, the membrane NADH oxidase is in isolated, partially purified or a recombinant form. Most particularly the membrane NADH oxidase is suitable for use in patients suffering from chronic fatigue having aberrant membrane NADH oxidase activity.

Yet a further aspect of the invention provides methods for identifying or determining the effect of a substance capable of inducing aberrant prion functioning and/or Aberrant Prion Disease, the method comprising (a) providing cells comprising constitutive ecto-nox proteins at their surface, (b) contacting the cells with a test-compound, and (c) determining whether or not aberrant ecto-nox proteins are generated.

In particular embodiments the methods according to this aspect of the invention comprise contacting the cells with an NADH containing hypotonic solution and/or an NADH containing isotonic solution. In further particular embodiments the substance is considered to be capable of inducing aberrant prions and/or APD when it is observed that the resulting NADH oxidase activity in the sample is higher under isotonic than under hypotonic conditions.

In particular embodiments of these methods according to the invention, NADH oxidase activity is determined by contacting the cells with a labeled substrate.

Yet a further aspect of the invention relates to methods of identifying a compound capable of reducing or inhibiting aberrant ecto-nox functioning in a patient, the method comprising contacting a sample of said patient with a test compound and determining the effect of said compound on the presence of aberrant ecto-nox proteins in said sample; in these methods, the presence of aberrant ecto-nox proteins is determined by a method comprising (1) contacting the sample with a colorimetric or luminometric substrate and NADH and (2) detecting NADH oxidase activity, wherein the intensity of the color or light is indicative of the presence of (aberrant) ecto-nox proteins in a sample. In particular embodiments the effect of the compound on NADH oxidase activity of the sample is compared determined based on comparison with the NADH activity of a sample of the patient in the absence of the compound.

### DETAILED DESCRIPTION

The present invention will be described with respect to particular embodiments, but the invention is limited only by the claims. The term “comprising” as used herein, implies that other elements or steps are not excluded. Unless specified differently, the use of an indefinite or definite article when referring to a singular noun (e.g. “a” or “an”, “the”), is intended to include reference to a plural of that noun. The terms first, second, third etc. as used herein, are used for distinguishing between steps or elements and do not necessarily imply a sequential or chronological order.

The following terms or definitions are provided to further the understanding of the invention.

### DEFINITIONS

The term “aberrant prion functioning” when referring to a patient as used herein refers to the fact that on the membrane surface of cells present in a sample of said patient are characterized by aberrant or activated NADH oxidase activity. More particularly reference is made to constitutive ecto-nox proteins which do not function as they do in healthy cells.

The term “Aberrant Prion Disease” as used herein refers to a disease characterized by the presence on the membrane surface of cells of the patient of aberrant or activated membrane NADH oxidase.

The presence of aberrant or activated membrane NADH oxidase can be determined by a number of methods including but not limited to the methods disclosed herein.

The term “chronic fatigue” as used herein refers to a clinically evaluated persisting or relapsing fatigue which is not the result of ongoing exertion, and is not substantially alleviated by rest.

The terms “Chronic Fatigue Syndrome” of “CFS” as referred to herein designates a condition which is diagnosed based on the following criteria (as developed by the U.S. Centers for Disease Control and Prevention in 1994):
1. Clinically evaluated, unexplained persistent or relapsing chronic fatigue that is of new or definite onset (i.e., not lifelong), is not the result of ongoing exertion, is not substantially alleviated by rest, and results in substantial reduction in previous levels of occupational, educational, social, or personal activities.
2. The concurrent occurrence of four or more of the following symptoms: substantial impairment in short-term memory or concentration; sore throat; tender lymph nodes; muscle pain; multi-joint pain without swelling or redness; headaches of a new type, pattern, or severity; unrefreshing sleep; and post-exertional malaise lasting more than 24 hours. These symptoms must have persisted or recurred during 6 or more consecutive months of illness and must not have predated the fatigue.

The term “processed tissue extract” as used herein refers to an extract of a mammalian tissue such as liver or kidney which has been processed (homogenization and optionally extraction of the protein fraction) and is suitable for therapeutic use. Processed liver extract is commercialized under the name of Hepapressin, Kutapressin® Nexavir® and Factor AF2.

“NADH oxidase activity” as used herein refers to the enzymatic transfer of electrons from reduced pyridine nucleotide (NADH) to molecular oxygen in the absence of added electron acceptors.

The term “ecto-nox protein” or “membrane NADH oxidase” as used herein refers to a cell-surface protein with both hydroquinone oxidase and protein-disulfide-thiol interchange activity. Constitutive ecto-nox proteins or prions are constitutively present in cellular membranes. They differ in this respect from mutated ecto-nox proteins present e.g. in cancer cells, such as t-nox.

The term “normal” when used in the context of ecto-nox protein or membrane NADH oxidase as used herein refers to the membrane NADH oxidase identifiable on normal, non-diseased cells and is characterized by the fact that it is heat sensitive (loss of activity when subjected to 70 degrees Celsius for 10 min.) and preferentially activated under hypotonic conditions.

The term “aberrant” when used in the context of ecto-nox protein or membrane NADH oxidase herein refers to an ecto-nox protein which does not function properly. In particular embodiments, the ecto-nox protein is in a permanently activated state and characterized by the fact that it is heat insensitive (retains activity when subjected to 70 degrees Celsius for 10 min.) and shows highest activity under isotonic conditions.

The term “ecto-nox protein modifying agent” as used herein refers to any compound capable of converting a normal ecto-nox protein (or membrane NADH oxidase) into an aberrant ecto-nox protein (or membrane NADH oxidase) or capable of inducing aberrant NADH oxidase activity in a sample.

The present invention is based on the observation that the symptoms of chronic fatigue and lack of energy reported by a number of patients and not attributable to an apparent cause are in many cases at least in part attributable to aberrant ecto-nox or membrane NADH oxidase functioning. The presence of aberrant membrane NADH oxidase prions at the membrane of a cell will result in excess NADH oxidase activity, such that excess electrons are transported out of the cell. This results in a shortage of electrons within the cell, leading to reduced ATP production. ATP is involved in numerous body processes as the energy molecule, so decreased ATP levels will have profound effects on the body, and more particularly depleted ATP levels will directly result in fatigue.

Accordingly, the inventors have identified a new class of patients characterized by aberrant ecto-nox function. As ecto-nox proteins have been identified as prion-like proteins these patients are generally referred to herein as suffering from Aberrant Prion Disease or APD. Accordingly, the present invention characterizes a new group of patients which are susceptible to treatment with compounds or compositions capable of reducing aberrant ecto-nox activity or reducing the presence of aberrant ecto-nox proteins. It is noted that in the art inappropriate NADH oxidase functioning has only been associated with cancer (Chueh et al., 2002, Biochemistry 41: 3732-3741), and this as a result of the present of mutated ecto-nox proteins.

In the context of the present invention, methods are provided to determine the presence of aberrant ecto-nox prions in a sample, more particularly a patient sample. These methods are based on structural, or functional properties of aberrant membrane NADH oxidase. More particularly it has been found that, contrary to normal functioning constitutive membrane NADH oxidase which has a cyclic oxidase activity, in particular embodiments, aberrant membrane NADH oxidase is permanently activated. This implies that it will react differently to inhibitors and/or stimulators.

According to particular embodiments, methods for detecting the presence of aberrant prions are provided, more particularly in a sample from a patient, which methods are based on the functional change of aberrant ecto-nox proteins compared to their normally functioning counterparts. The presence of aberrant functioning ecto-nox proteins can result in increased or decreased NADH oxidase activity in the sample of the patient, compared to a sample of a healthy control. In more particular embodiments the aberrant ecto-nox proteins are permanently activated ecto-nox proteins (e.g. as a result of the contact of the patient with metals). In more particular embodiments, this involves determining differences in NADH oxidase activity, optionally in the presence of specific agents or modulators.

Methods for determining NADH oxidase activity are known in the art. Such methods include methods based on determining the rate of disappearance of NADH, the appearance of NAD, or the rate of appearance or disappearance of a reaction product or reactant, respectively, either directly or indirectly.

In particular embodiments, NADH oxidase activity is measured by detecting an electron acceptor in the assay for NADH oxidase, conveniently an ascorbate radical, where one may follow the rate of disappearance of the ascorbate radical under the conditions of the assay. One can measure the reduction of ascorbate free radical at 265 nM employing a spectrophotometer as described by Winkler, Biochim. et Biophys. Acta 925:258-264 (1987).

According to particular embodiments, the methods for determining the activity of membrane NADH oxidase envisaged herein involve the reduction of an indicator substrate, such as chemiluminogenic, colorimetric or fluorogenic substrates, by a dehydrogenase, whereby NADH acts as a co-enzyme. Typically, the substrate is a chemiluminescent compound such as an acridinium derivative or a colorimetric substrate such as a tetrazolium dye. Examples of acridinium derivatives include but are not limited to acridinium esters such as Polysubstituted Aryl Acridinium Esters (PAAE; U.S. Pat. Nos. 4,745,181; 4,918,192; and 5,110,932), functionalized Hydrophilic PAAE (U.S. Pat. No. 5,656,426) or 9,10-dimethyl biacridiniumdinitrate (bis-N-methylacridinium nitrate or lucigenin) and concentration ranges of 0.1-10 mM, preferably 0.5-2 mM.

Examples of tetrazolium dye include 2-(p-nitrophenyl)-3-(p-iodophenyl)-5-phenyltetrazolium chloride (hereinafter abbreviated as INF), 3,3′-(3,3′-dimethoxy-4,4′-diphenylene)bis(2-(p-nitrophenyl)-5-phenyltetrazolium chloride), 2-(4′,5′-dimethyl-2′-thyazolyl-3,5-diphenyltetrazolium bromide (hereinafter abbreviated as MTT) and the like. A typical concentration range of tetrazolium dye in the analytical reagent is 0.1-10 mM, preferably 0.5-2 mM.

In particular embodiments the methods for detecting NADH oxidase activity are based on the methods described in U.S. Pat. No. 5,306,624. This patent describes a method for detecting viable cells comprising the steps of: a) admixing an effective detection amount of an energy-emitting nonhazardous probe with the suspension to form an admixture, wherein the emission of energy from the probe is proportional to and activated by a stimulant; b) exposing the admixture to an effective triggering amount of a probe-trigger, wherein the probe-trigger interacts with the viable cells in the suspension to generate the stimulant in an amount proportional to the number of the viable cells; c) maintaining the admixture under physiological reaction conditions and for a period of time sufficient for activation of the energy-emitting nonhazardous probe; and d) detecting the emission of energy from the probe. Similarly the method can be used for the detection of superoxide radical formation which is the stimulant. Most particularly, the probe-trigger is NADH. An energy-emitting non-hazardous probe is a light-emitting non-hazardous probe such as a chemiluminogenic probe or an otherwise luminescent probe. A preferred chemiluminogenic probe is lucigenin, lophine, luminol, a dioxetane or acridinium ester.

In particular embodiments of the detection methods according to the invention exogenous dehydrogenases are added. Dehydrogenases which can be used in the context of the present invention include but are not limited to amino acid dehydrogenases (hereinafter abbreviated as AADH), AdH and the like having a potent specificity to NAD or NADH. For example, AADH may include alanine dehydrogenase (hereinafter abbreviated as AlaDH), leucine dehydrogenase (hereinafter abbreviated as LeuDH), glutamate dehydrogenase and the like. Any ADH, for example, those originated from baker's yeast, a microorganism belonging to genus of Zymomonas and the like, can be used, and their origin is not limited. Typical concentration range of these dehydrogenases is 0.01-1000 units/ml, preferably 0.1-100 units/ml. One unit of dehydrogenase means the quantity of enzyme which can oxidize 1 μmol of corresponding substrate per minute at pH 9.0 and 30° C. When AADH is used as dehydrogenase, a specific amino acid corresponding to a specific AADH should be used. Alcohol such as ethanol is used as a substrate for ADH. Typical concentration range of substrates for dehydrogenases in the analytical reagent is 1-1000 mM, preferably 10-100 mM. Any buffer solution having buffer action within neutral pH range, for example, phosphoric acid buffer, triethanol buffer and the like can be used. A typical pH range of the buffer is 5.0-10.0, preferably 7.0-9.0. A typical concentration range of the buffer in the analytical reagent is 10-1000 mM, preferably 50-500 mM.

In particular embodiments of the methods of the present invention, the NADH oxidase activity of the sample is compared to that of a control sample. Typically the control sample is a sample known not to contain aberrant NADH oxidase. More particularly, where the test sample is a patient sample comprising cells, the control sample can be a sample comprising cells from a healthy control.

In further particular embodiments the methods for determining aberrant NADH oxidase activity envisaged herein involve determining the activity of membrane NADH oxidase in a sample in the presence of an agent. Such an agent is typically an NADH oxidase activator or NADH oxidase inhibitor.

Typical membrane oxidase activators will induce activation of normal non-aberrant membrane NADH oxidase; However, where the membrane NADH oxidase is aberrant, i.e. permanently activated, contacting with an activating agent will not significantly influence NADH activity. Accordingly, this makes it possible to determine whether or not the sample contains aberrant membrane NADH oxidase proteins. Examples of NADH activators in normal cells include hypotonic stress, . . . etc.

Typically, normal non-aberrant membrane associated NADH oxidase becomes activated when mammalian cells are subjected to hypotonic stress. Extracellular plasmamembrane associated NADH oxidase exerts cell volume control. When mammalian cell types are subjected to hypotonic stress, they will tend to counteract volume increase part of which mechanism proceeds through activation of the plasmamembrane NADH-oxidase. Therefore, when a hypotonic solution of NADH and a chemiluminogenic substrate such as lucigenin are added to otherwise normal mammalian cells, NADH-oxidase will be activated and a chemiluminescent response will be obtained. It is noted however that in samples where the concentration of mammalian cells is relatively low, this effect may not be measurable. An isotonic NADH solution will not induce a chemiluminescent response in mammalian cells with normal, non-aberrant NADH oxidase. It has been observed however that in mammalian cells with aberrant NADH oxidase, or where the membrane NADH oxidase has been activated, the contacting of the cells with a hypotonic NADH solution will result in a faster and more significant increase of NADH oxidase activity compared to when the cells are contacted with an isotonic NADH solution.

Examples of NADH inhibitors are known in the art and include anthracyclines such as adriamycin and adriamycin conjugates, N-Acylated catecholmethylamines, lipophilic fatty acid amides of catecholmethylamines, compounds used in the treatment of malaria, such as primaquine, quinacrine, chloroquine; quinine.

In particular embodiments the methods of the present invention comprise determining the presence of aberrant membrane NADH oxidase in a sample by contacting the sample with an NADH oxidase activator and determining the effect of the NADH oxidase activator on the NADH oxidase activity (or another parameter related thereto) of the sample. A lack of response to an NADH oxidase activator is indicative of the presence of aberrant NADH oxidase.

In further particular embodiments, methods of the present invention comprise determining the presence of aberrant membrane NADH oxidase in a sample by contacting the sample with an isotonic and a hypotonic NADH solution, and determining the effect on NADH oxidase activity (or another parameter related thereto) of the sample. The effect of administering NADH under hypotonic or isotonic conditions can be monitored both in volume (absolute increase/decrease of NADH activity) and/or in time (kinetics of NADH oxidase activity).

In more particular embodiments the sample of interest is contacted with a solution of 5.10⁻⁴M NADH either in aqua distillata or in isotonic water, followed by the detection of NADH oxidase activity (e.g. based on luminescent substrate).

In further particular embodiments of the methods of the present invention cyanide is used to eliminate mitochondrial oxidase activity.

In particular embodiments, the detection of the presence of aberrant ecto-nox proteins, such as in sample is based on structural properties of the aberrant proteins compared to the proteins in their normal configuration. This can be detected using specific binding agents which differentiate between normal and aberrant ecto-nox proteins. In particular embodiments the detection of aberrant prions is based on an immunological detection. Antibodies, specifically recognizing the activated form of membrane NADH oxidase, optionally tagged with a label, can be used to identify the presence of aberrant ecto-nox proteins. Suitable labels are considered labels which ensure a detectable signal including but not limited to radioisotopes, enzymes, fluorescers, chemiluminescers. The term antibodies as used herein, refers to any molecule derived from a classical antibody or comprising one or more of its antigen-binding structures.

In one aspect the present invention provides methods and tools for determining aberrant prion functioning in a patient. More particularly the invention provides methods for the diagnosis of Aberrant Prion Disease. More particularly methods and tools are provided to determine whether a patient is suffering from aberrant prion functioning or Aberrant Prion Disease. In particular embodiments these methods comprise determining, in a sample of the patient, whether or not aberrant prions are present. Methods for determining the presence of aberrant prions in a sample are described in detail above. Such methods are preferentially carried out on samples such as blood or saliva samples.

Further particular embodiments of this aspect of the invention provide methods which allow the identification of a patient susceptible to treatment with a composition reducing the presence and/or activity of aberrant prions, This allows a more efficient treatment of patients diagnosed with a particular disease, more particularly a diseases such as fybromyalgia, chronic fatigue syndrome, undefined infectious diseases, immunological disorders, nervous system disorders, intoxications, defective woundhealing processes, gulf-war syndrome, etc. Indeed, in particular embodiments the invention provides methods for determining whether or not a sample of a patient is characterized by aberrant prion functioning and, in the positive, using a sample of the patient to identify a compound capable of reducing aberrant prion functioning.

Particular embodiments of the methods of the present invention include methods for determining, in a patient suffering from general complaints of fatigue, whether or not the patient is characterized by aberrant prion functioning. In further particular embodiments the invention provides methods determining, in a patient diagnosed with chronic fatigue, whether or not he/she is suffering from aberrant prion functioning or Aberrant Prion Disease. Most particular embodiments relate to methods for determining, in a patient diagnosed with Chronic Fatigue syndrome (CFS), whether or not he/she is suffering from aberrant prion functioning or Aberrant Prion Disease. Similarly methods are provided for determining the susceptibility of a patient diagnosed with chronic fatigue, more particularly a patient diagnosed with CFS, for the treatment with a composition reducing the presence and/or activity of aberrant prions. More particularly methods are provided for determining the susceptibility of a patient diagnosed with a disease selected from fybromyalgia, chronic fatigue syndrome, undefined infectious diseases, immunological disorders, nervous system disorders, intoxications, defective woundhealing processes, gulf-war syndrome to the treatment with a composition reducing the presence and/or activity of aberrant prion functioning. More particularly the methods comprise determining which composition would be suitable to reduce the presence and/or activity of aberrant prion function in said patient.

The methods for diagnosing ABS or other diseases envisaged herein are in particular embodiments carried out in vitro on a sample. The sample is typically a physiological sample which may be a tissue sample, plasma membrane fragments, lysate, serum, urine, saliva or other convenient physiological fluid.

As detailed above such methods may involve comparison between the test sample of the patient and a control sample which is known to contain only normal membrane NADH oxidase. Such methods may further include the step of adding agents to the sample to determine the effect of the agent on the sample. In particular embodiments, the methods comprise adding NADH to the sample either in isotonic or hypotonic solution.

The methods of diagnosing aberrant prion functioning or Aberrant Prion Disease allow for a better recognition of the underlying physiological actuator of the disease, thereby allowing more efficient treatment. This will make it possible to identify which patients will benefit from treatment with compounds or compositions which reduce the presence and/or activity of aberrant ecto-nox proteins (further detailed below).

The identification of a link between abnormal membrane NADH oxidase and Aberrant Prion Disease also allows the identification of agents which induce abnormal NADH oxidase phenotype in cells, and thus may induce aberrant prion functioning and Aberrant Prion Disease. Indeed it is of interest to obtain a functional screen in which samples or agents can be tested for their APD-inducing effect. Accordingly, yet another aspect of the present invention relates to methods for identifying compounds or conditions capable of inducing APD in a patient. This allows the identification of environmental hazards and toxic wastes.

In particular embodiments the methods according to this aspect of the present invention involve contacting a sample containing essentially only normally functioning membrane NADH oxidase with an agent or composition and determining whether aberrant membrane NADH oxidase is generated. In more particular embodiments, determining whether aberrant membrane NADH oxidase is generated is ensured by subjecting the cells to particular conditions which allow detection of aberrant membrane NADH oxidase. Methods for detecting the presence of aberrant membrane NADH oxidase are described in detail above and will not be repeated here.

In further particular embodiments the capacity of a condition, compositon or agent capable of inducing the formation of aberrant membrane NADH oxidase is determined by comparing the effect of the condition, compositon or agent of interest on the NADH oxidase activity of a sample to that of a (positive and/or negative) control sample, e.g. a sample known to comprise only normally functioning membrane NADH oxidase (negative control). In such embodiments the determination of a difference between the test sample and the control is indicative of the ability of the condition, composition or agent to induce aberrant membrane NADH oxidase activity, more particularly to induce APD.

In further particular embodiments the capacity of a condition, composition or agent capable of inducing the formation of aberrant membrane NADH oxidase is determined by comparing the NADH oxidase activity of a sample to which the condition, composition or agent of interest has been added to a comparable sample to which the condition, composition or agent has not been added. In yet further particular embodiments the effect of the addition of the condition, composition or agent under hypotonic and isotonic conditions is compared.

Methods according to this aspect of the present invention are particularly suitable for identifying causative agents, where an abnormal distribution of aberrant prion functioning or APD within a cell population or geographic region is observed or in the context of quality control. Such methods can be applied for determining the capacity of any type of condition, composition or agent to induce APD, such as, but not limited to (waste or drinking) water, industrial exhausts, raw materials, chemicals used in production etc.

A further aspect the invention relates to methods and tools for the identification of compounds and compositions suitable for the treatment or prevention of aberrant prion functioning or Aberrant Prion Disease. Such methods include, but are not limited to screening methods which involve determining the ability of compounds and/or compositions to reduce the activity and/or presence of aberrant ecto-nox prions. Methods for determining the activity and/or presence of aberrant ecto-nox prions are described in detail herein and will not be repeated here. The methods according to this aspect of the invention can make use of any one of the methods for identifying aberrant ecto-nox proteins (such as but not limited to methods based on determining NADH oxidase activity) disclosed herein or known in the art.

In particular embodiments, the methods for identifying a compound or composition for use in the treatment or prevention of aberrant prion functioning in a patient or of APD involve providing a sample containing aberrant prions, contacting the sample with a test compound and determining whether or not the test compound is capable of reducing the activity and/or presence of aberrant ecto-nox proteins.

In further embodiments, the methods for identifying a compound or composition for use in the treatment or prevention of aberrant prion functioning or APD involve the steps of providing a sample containing only constitutive ecto-nox proteins, contacting the sample with (a) a compound or condition capable of inducing aberrant ecto-nox proteins and (b) a test compound and determining whether or not the test compound can reduce or prevent the development of aberrant ecto-nox proteins.

In further particular embodiments the methods for identifying compounds or compositions for use in the treatment or prevention of APD involve the steps of providing a sample containing normally functioning constitutive ecto-nox or membrane NADH oxidase proteins, contacting the sample with (a) a compound or condition capable of inducing aberrant ecto-nox proteins and (b) a test compound and determining whether the test compound affects the ability of the ecto-nox proteins in the sample to react to hypotonic or isotonic conditions. Typically it is observed that the NADH oxidase activity of cells comprising aberrant NADH oxidase is higher when NADH is added in an isotonic solution than when NADH is added in a hypotonic solution. Accordingly, a ratio of NADH oxidase activity isotonic/hypotonic greater than one (>1) is indicative of aberrant NADH oxidase activity. Accordingly, where the test compound or composition induces a ratio higher than one, it is considered to induce aberrant NADH oxidase activity.

The effect of the compound or composition can be seen as a quantitative effect and/or a kinetic effect. In particular embodiments a compound capable of delaying the kinetics and/or reducing the extent of NADH oxidase activity is a compound capable of reducing the activity and/or presence of aberrant ecto-nox proteins.

Compounds capable of inducing aberrant NADH oxidase activity include but are not limited to particular heavy metals.

It will be understood to the skilled methods that these methods can be used to identify either one compound or composition or combinations of compounds or compositions and thus the step of contacting the sample with the test compound or composition can comprise contacting the sample with one or more test compounds or compositions or one or more conditions simultaneously or sequentially.

In particular embodiments, methods for identifying a compound or composition for use in the treatment or prevention of aberrant prion functioning or APD involve the steps of:
(1) Admixing an appropriate volume of a sample comprising (mammalian cells carrying) an aberrant NADH-oxidase with an appropriate volume of solution of a test compound or composition;
(2) Adding an appropriate amount of a colorimetric, fluorogenic or chemiluminescent substrate;
(3) Adding an appropriate volume of an NADH containing solution;
(4) Measuring the colorimetric, fluorimetric or chemiluminometric response upon the addition of the NADH containing solution.
(5) Comparing the result obtained in (4) with the result obtained with a control sample to which the test compound or composition has not been added (addition of equal volume of solvent).

In further particular embodiments the methods for identifying a compound or composition for use in the treatment or prevention of APD involve the steps of.
(1) Admixing an appropriate volume of a sample comprising (mammalian cells carrying constitutive membrane NADH oxidase,
(2) inducing aberrant NADH-oxidase with a compound capable of inducing aberrant NADH oxidase,
(3) contacting the sample with an appropriate volume of solution of a test compound or composition;
(4) Adding an appropriate amount of a colorimetric, fluorogenic or chemiluminescent substrate;
(5) Adding an appropriate volume of an NADH containing solution;
(6) Measuring the colorimetric, fluorimetric or chemiluminometric response upon the addition of the NADH containing solution.
(7) Comparing the result obtained in (6) with the result obtained with a control sample to which the test compound or composition has not been added (addition of equal volume of solvent).

In particular embodiments the sample is a patient sample and the methods allow the identification of a suitable therapeutic compound for the treatment of said patient.

In particular embodiments of the methods above, the NADH containing solution is added as either a hypotonic or an isotonic solution and the difference observed under these conditions (optionally compared to control) is a measure for the ability of the test compound or condition to reduce the presence and/or activity of aberrant NADH oxidase activity, Typically it is observed that the NADH oxidase activity of cells comprising aberrant NADH oxidase is higher when NADH is added in an isotonic solution than when NADH is added in a hypotonic solution. Accordingly, a ratio of NADH oxidase activity isotonic/hypotonic greater than one (>1) is indicative of aberrant NADH oxidase activity. Compounds capable of reducing the ratio to less than one or delaying the reaction to isotonic NADH are considered to be capable of reducing the presence and/or activity of aberrant NADH and thus suitable for the treatment of APD.

According to this embodiment test compound or compositions are identified based on their effect on aberrant NADH-oxidase, whereby the ability of the compound to affect NADH-oxidase activity in the cells (measured by detection of the colorimetric, fluorimetric or chemiluminometric response) is indicative of the ability of the test compound or composition to reduce or prevent APD. In further particular embodiments the screening can comprise comparing the effect of the test compound or composition to the NADH activity in a control sample not comprising aberrant NADH oxidase but constitutive NADH oxidase and/or to a control sample (with or without aberrant NADH) but to which a composition known to affect activated NADH oxidase is added. Most particularly the effect on the compound on NADH oxidase under hypotonic vs. isotonic conditions is determined.

Yet a further aspect of the present invention relates to methods of treating and/or reducing the symptoms of a patient suffering from aberrant prion functioning or Aberrant Prion Disease and the provision of compounds and compositions for use in therein.

In particular embodiments the methods of treatment involve inhibiting and/or reducing aberrant membrane NADH oxidase activity. This can be ensured in different ways.

In particular embodiments of the invention aberrant membrane NADH oxidase is inhibited by inhibiting or reducing the association of the aberrant membrane NADH oxidase with the membrane. This inhibition can be either specific or non-specific. For instance, it is envisaged that specific compounds binding to aberrant membrane NADH oxidase can interfere with the association of aberrant membrane NADH oxidase to the membrane. Examples of such compounds include, but are not limited to specific binding agents such as (monoclonal) antibodies and derivatives thereof. Antibodies capable of interfering with the binding of NADH oxidase to the membrane can be developed using standard techniques.

Examples of non-specific agents which interact with the association of NADH oxidase with the membrane include solvents or detergents, more specifically biocompatible such as, but not limited to DMSO, long alkyl esters of arginine, Polyurethane block copolymers etc.

The effect of compounds capable of interfering with the association of aberrant membrane NADH oxidase with the membrane can be observed in different ways. The reduction can be detected at the cellular level, where a reduction in NADH oxidase activity is observed. However it has been observed that such compounds and compositions induces at least a temporary increase NADH activity in the urine of treated patients, This may be due to shedding of the aberrant membrane NADH oxidase.

In a further particular embodiment of the methods of treating or preventing aberrant prion functioning or APD in a patient according to this aspect of the invention aberrant membrane NADH oxidase activity is inhibited and/or reduced by contacting aberrant NADH oxidase with compounds or compositions which compete with aberrant membrane NADH oxidase, for the membrane NADH oxidase substrate. More specifically it is envisaged to reduce aberrant NADH oxidase activity in a patient by administering constitutive or normal (membrane) NADH oxidase. Membrane NADH oxidase is present in tissue extracts, more particularly liver or kidney extracts. Examples of commercially available extracts include Kutapressin® and Nexavir®.

In particular embodiments of the invention the use of a purified NADH oxidase is envisaged. Membrane NADH oxidase has been described to consist of a complex of at least three peptide chains with molecular weights between 30 kDa and 75 kDa. These can be purified using techniques known to the skilled person such as, but not limited to treatment with detergents, fractionation on an affinity and/or gel filtration and anion exchange chromatography.

In yet further particular embodiments of the invention aberrant NADH oxidase activity is reduced or inhibited by the administration of NADH oxidase inhibitors. Suitable membrane NADH oxidase inhibitors have been described above and include but are not limited to anthracyclines such as adriamycin and adriamycin conjugates, N-Acylated catecholmethylamines, lipophilic fatty acid amides of catecholmethylamines, compounds used in the treatment of malaria, such as primaquine, chloroquine, quinine, quinacrine.

In particular embodiments the methods of treatment described above envisaged above comprise the administration of an inhibitor of aberrant membrane NADH in combination with another active ingredient. The (aberrant) membrane NADH oxidase inhibitor and other active agent may be combined together, mixed or reacted, either covalently, i.e. conjugated, or noncovalently, or may be administered simultaneously.

The present invention identifies aberrant prion function as an important underlying physiological characteristic of patients suffering from chronic fatigue and lack of energy and provides compositions for use in the treatment of such patients. It is advisable to determine whether or not the patient suffers from ABS prior to treating the patient using the methods described herein. Accordingly, particular embodiments of the invention involve the steps of a) determining whether or not the patient is suffering from APD and b) treating the patient according to one or more of the methods described herein.

As detailed above, patients suffering from chronic fatigue are in practice diagnosed with differing diseases. Chronic fatigue is characterized as recurring fatigue with no evident cause which is not alleviated by sleep. More particularly this does not relate to cancer patients or patients undergoing chemotherapy. The present invention demonstrates that in at least a subpopulation of patients suffering from chronic fatigue, aberrant prion function is a mediator of the disease and treatment according to the invention can be effective. Accordingly a particular embodiment of the present invention relates to methods for treating a patient suffering from chronic fatigue which is characterized by Aberrant Prion Disease, which involve administration of one or more of the compounds detailed above. In further particular embodiments of the invention the above methods are used for treating a patient diagnosed with Chronic Fatigue Syndrome (CFS) and characterized by Aberrant Prion Disease. By identifying the population susceptible to this type of treatment, the treatment is more effective and avoids frustration in non-responders. Most particularly patient groups in which medication suitable for the treatment of APD can be used with more than 75% efficiency can be identified.

A further aspect of the invention relates to the development of new therapeutic strategies and/or more efficient therapeutic strategies for the treatment of patients characterized by chronic fatigue and/or diagnosed with chronic fatigue syndrome (CFS). As detailed above, a significant percentage of these patients suffer from APD, warranting treatment with a compound or composition reducing or inhibiting aberrant prions. Thus, despite the fact that such treatments will not be effective in all patients suffering from chronic fatigue and/or diagnosed with CFS, the percentage of efficacy is envisaged to be sufficiently high to justify treating the patient with compositions or compounds capable of reducing aberrant prion disease, even where the latter has not been specifically determined. Accordingly, the present invention further provides methods and tools for the treatment of patients suffering from chronic fatigue and/or diagnosed with CFS, which involve administering to the patient a compound capable of modulating an aberrant prion.

The present invention describes a new mechanism underlying typical symptoms of a disease and opens up the potential to develop new therapies based on this mechanism. The application thus focuses on the one hand on the application of this therapy to a group of patients which has not been previously identified as such and on the other hand on the identification of suitable compounds for treatment based on the identification of this mechanism.

### Examples

1. Determination of Aberrant Membrane NADH Oxidase Activity in Samples of Patients Suffering from Chronic Fatigue Compared to Control.

The membrane NADH oxidase activity of four patients suffering from chronic fatigue was compared to that of a control not suffering from fatigue.

100 μl saliva obtained from patients suffering from chronic fatigue or from the control was pipetted into eppendorf tubes. To this volume 100 μl lucigenin (10⁻³ M in aq. dist) or lucigenin (10⁻³ M in isotonic water) were added. Next eppendorf tubes were placed into scintillation counting vials (10 ml) and a chemiluminescence reaction was started by the addition of either hypotonic (5.10⁻⁴ M NADH dissolved in aq. dist.) or isotonic (5.10⁻⁴M NADH dissolved in isotonic water (Sigma)) NADH solution. The luminescence (CPM tritium channel) was observed after 5 minutes.

### Results:

| | CPM hypotonic | CPM isotonic |
|---|---|---|
| Patient 1 RG | 15915 | 42536 |
| Patient 2 RB | 700 | 3590 |
| Patient 3 HB | 3593 | 7987 |
| Patient 4 HL | 1131 | 2650 |
| Control CR | 238 | 296 |

It was observed that the saliva from patients suffering from chronic fatigue showed a significantly higher luminescence response than the saliva from control patients. This suggests that the saliva from these patients contains significantly higher NADH oxidase activity than that of the control patient.

Moreover, it was observed that the effect of the addition of NADH under hypotonic conditions in the control patient could not be observed as it remained under the baseline value. The saliva samples of the patients suffering from chronic fatigue however, showed faster luminescence kinetics and a higher light output under isotonic than under hypotonic conditions. This suggests a faster and more pronounced increase in NADH activity upon contacting with NADH under isotonic conditions for patients with chronic fatigue.

2. Effect of Nexavir on Induced Aberrant NADH Oxidase Activity

K562 cells were cultured in RPMI medium supplemented with 10% FCS and antibiotics (Pen-Strep) in the presence of different concentrations of HgCl₂. After overnight culture in Falcon flasks in a total volume of 10 ml medium with and without Nexavir (250 μg/ml) each containing HgCl₂ at a final concentration of 100 μg/ml, 10 μg/ml, 1 μg/ml, 100 ng/ml, 10 ng/ml and blank medium respectively, cells were harvested, centrifuged, washed and resuspended in 1 ml PBS (pH 7.4).

Next, 100 μl washed cells were pipetted into 1 ml eppendorf tubes and placed in scintillation counter vials.

After the addition of 100 μl lucigenin (10⁻³ M in aq. dist.) and 800 μl NADH (5.10⁻⁴M in aq. dist.) the luminescence reaction was observed in a Beckman 6500 Scintillation counter producing the following counts (CPM measured in the Tritium channel) after 5 minutes:

| | | Effector | |
|---|---|---|---|
| Concentration | | HgCl₂ | HgCl₂ + Nexavir (250 μg/ml) |
| 100 | μg/ml | 914 | 673 |
| 10 | μg/ml | 68 | 4256 |
| 1 | μg/ml | 64 | 8584 |
| 100 | ng/ml | 4930 | 15438 |
| 10 | ng/ml | 29331 | 1418 |
| 0 | | 2541 | 2066 |

Results show
a protective effect (higher counts) of Nexavir on the toxic effect of mercury at concentrations of 1 μg/ml-10 μg/ml.
a protective effect (lower counts) of Nexavir on the aberrant NADH-oxidase inducing effect of mercury at concentrations of 10 ng/ml-100 ng/ml.

3. Stimulation of Constitutive NADH Oxidase by DMSO and Heparin.

K562 cells cultured in RPMI 1640 Medium supplemented with 10% FCS and 50 ng/ml of HgCl₂ were harvested, washed and resuspended in PBS pH 7.4. To 100 μl cell suspension, 100 μl of a serial diluted DMSO stock solution (99%) or Heparin solution (100 IE/UI/ml) in PBS were added. Next 100 μl lucigenin (10⁻³ M) were added and the reaction started by the addition of 900 μl hypotonic NADH-solution (5.10⁻⁴M) or isotonic NADH-solution (5.10⁻⁴M). The luminescence (CPM) was recorded after 5 minutes.

| DMSO/water | CPM | Heparin | CPM |
|---|---|---|---|
| 50% | 97262 | 50% | 17740 |
| 25% | 28105 | 25% | 15303 |
| 12.5% | 17764 | 12.5% | 14082 |
| 6.25% | 12929 | 6.25% | 10372 |
| 3.12% | 8906 | 3.12% | 9192 |
| 0 | 8494 | 0 | 7394 |

Whereas the culturing of K562 cells in medium containing 50 ng/ml of HgCl₂ over a long period (72 hrs) induces the formation of cell surface associated aberrant NADH oxidase, a significant concentration-dependent increase in chemiluminescence response is observed after the addition of DMSO or Heparine and hypotonic NADH, which was not observed after the addition of isotonic NADH, indicating a conformational change of the aberrant type NADH oxidase.

4. Identification of Factors Capable of Inducing Aberrant Membrane NADH Oxidase

K562 cells were grown in RPMI 1640 medium supplemented with 10% FCS and 1 μg/ml of Thimerosal, HgCl₂, NiCl₂, CdCl₂ and Pb(NO₃)₂. After overnight culture, cells were harvested, washed and resuspended in PBS, pH 7.4.

To 100 μl cell suspension, 100 μl isotonic or hypotonic lucigenin 10⁻³M were added and the luminescence reaction was started by the addition of 900 μl isotonic or hypotonic NADH (5.10⁻⁴ M) solution.

| | CPM NADH | CPM NADH | CPM |
|---|---|---|---|
| | isotoon | hypotoon | iso/hypo |
| Thimerosal | 2380 | 3338 | 0.71 |
| HgCl₂ | 101 | 18 | 5.61 |
| NiCl₂ | 913 | 860 | 1.06 |
| CdCl₂ | 114 | 376 | 0.30 |
| Pb(NO₃)₂ | 1402 | 817 | 1.71 |
| 0 | 4018 | 10248 | 0.39 |

Higher counts obtained with HgCl2, NiCl2 and Pb(NO3)2 under isotonic conditions suggest induction of aberrant NADH-oxidase.

5. Evolution Shedded NADH-Oxidase in Urine of CFS Patient Before and after DMSO Treatment

Luminescence: 100 μl urine+100 μl lucigenin 10-3 M+800 μl NADH (ISO/HYPO at 5.10⁻⁴M)

| Date | condition | CPM (isotonic/hypotonic) |
|---|---|---|
| 12/02 | before | 316/55 |
| 13/02 | after 1 hr. DMSO | 178/28 |
| 13/02 | after 2 hrs. DMSO | 1008/119 |
| 17/02 | | 577/31 |
| 18/02 | | 7/119 |

During DMSO treatment isotonic NADH induced lucigenin-dependent chemiluminescence increases. Increase in isotonic NADH induced chemiluminescence in urine may indicate shedding of aberrant NADH oxidase.

1. An in vitro method of determining the presence of aberrant ecto-nox functioning in the sample of a patient, said method comprising the steps of:
(a) contacting a cell-containing sample of the patient with an NADH solution and a colorometric or luminometric substrate,
(b) detecting NADH oxidase activity, and
(c) determining the presence of aberrant ecto-nox functioning based on the detected NADH oxidase activity.

2. The method of claim 1, which further comprises comparing NADH oxidase activity in the sample to that of a control sample.

3. The method of claim 1, which comprises contacting the sample with a hypotonic NADH solution and/or an isotonic NADH solution.

4. The method of claim 3 which comprises contacting the sample with a hypotonic NADH solution and an isotonic NADH solution and comparing the effect of the NADH hypotonic solution and the NADH isotonic solution on NADH oxidase activity.

5. The method of claim 1, which is a method of determining whether the patient suffers from Chronic Fatigue Syndrome.

6. The method of claim 1, which is a method for determining whether or not a patient diagnosed with a particular disease is susceptible to treatment with a compound capable of reducing or inhibiting aberrant ecto-nox functioning.

7. The method of claim 6, which is a method for determining whether or not a patient suffering from chronic fatigue is susceptible to treatment with a compound capable of reducing or inhibiting aberrant ecto-nox functioning.

8.-13. (canceled)

14. A method for identifying or determining the effect of a substance capable of inducing aberrant ecto-nox functioning, the method comprising:
(a) providing cells comprising constitutive ecto-nox proteins at their surface,
(b) contacting the cells with a test-compound, and
(c) determining whether or not aberrant ecto-nox proteins are generated by contacting the cells with an NADH containing hypotonic solution and a colorimetric or luminometric substrate optionally by contacting the cells with an NADH-containing hypotonic solution.

15. A method of identifying a compound capable of reducing or inhibiting aberrant ecto-nox functioning in a patient, the method comprising:
(a) contacting a sample of said patient with a test compound, and
(b) determining the effect of said compound on the presence of aberrant ecto-nox proteins in said sample, whereby the presence of aberrant ecto-nox proteins is determined by a method comprising:
(i) contacting the sample with a colorimetric or luminometric substrate and NADH
(ii) detecting NADH oxidase activity, wherein the intensity of the color or light is indicative of the presence of (aberrant) ecto-nox proteins in a sample.
